(19) United States
(12) Patent Application Publication
(10) Pub. No.: US 2016/0326226 A1
(43) Pub. Date: Nov. 10, 2016

(54) RETICULOCYTE GROWTH FACTOR AND PREPARING METHOD AND APPLICATION THEREOF

(71) Applicant: NANJING BIYOUKANG BIOTECHNOLOGY CO., LTD., Nanjing, Jiangsu (JP)

(72) Inventors: YIFENG JIN, JIANGNINGQU (CN); QI WU, JIANGNINGQU (CN); TAIPING ZHANG, JIANGNINGQU (CN); XIAOYAN MA, JIANGNINGQU (CN); JI XUAN, JIANGNINGQU (CN)

(21) Appl. No.: 15/036,075

(22) PCT Filed: Jan. 27, 2014
(86) PCT No.: PCT/CN2014/071586

§ 371 Date: May 12, 2016

(30) Foreign Application Priority Data
Jan. 24, 2014 (CN) 201410036118.X

(51) Int. Cl. C07K 14/475; A61K 38/18

(52) U.S. Cl. CPC C07K 14/475; A61K 38/18; A61K 38/1816

(57) ABSTRACT

A Reticulocyte Growth Factor (RGF) and a preparing method and application thereof is provided. The factor is separated and purified from impurities of α₁-antitrypsin (from human plasma), and the fragment of a precursor of the antitrypsin is determined, and has very high function of promoting the generation of the reticulocyte of mice. Monoclonal antibody of EPO is not combined with the reticulocyte growth factor, which is labeled with I¹²⁵, after the reticulocyte growth factor is combined with surface receptors of mouse bone marrow cells, the EPO cannot compete with the reticulocyte growth factor. The EPO and RGF are labeled with different fluorochromes, and it is proved that they have different receptors.

### CROSS-REFERENCE TO RELATED APPLICATIONS

This application claims priority to PCT Application No. PCT/CN2014/071586, having a filing date of Jan. 27, 2014, based off of Chinese Application No. 201410036118.X, having a filing date of Jan. 24, 2014 the entire contents of which are hereby incorporated by reference.

### SEQUENCE LISTING

This application include a separate sequence listing in compliance with the requirements of 37 C.F.R. §§1.824(a)(2)-1.824(a)(6) and 1.824(b), submitted under the file name “51605_Sequence_Listing_AS-FILED”, created on Apr. 20, 2016, having a file size of 6 KB, the contents of which are hereby incorporated by reference.

### FIELD OF TECHNOLOGY

The following relates to the field of medical biotechnology, and in particular relates to a reticulocyte growth factor and a preparing method and application thereof.

### BACKGROUND

At present, the most efficient medicine for clinically treating severe anemia is Erythropoietin (rhu-EPO). Since the recombinant human (rhu) EPO of the Amgen Company in America was approved by FDA in 1989, the rhu-EPO has been generally commonly accepted, and has developed to the most successful example in DNA recombinant medicine. EPO is one unique special red cell increasing factor in generation of red cells as so far. Although it is reported that Stem Cell Growth Factor (SCF) and Colony Stimulating Factors (CSF_{S}) also have a promoting action for the red cells, their functions are diversified and not exclusive. At present, no new medicine for treating the severe anemia is found yet.

### SUMMARY

An aspect relates to providing a new cell growth factor—Reticulocyte Growth Factor (RGF), and a preparing method and application thereof.

In a research process of embodiments of the present invention, in kidney cell culture fluid, proteins with erythropoiesis activity exist in both human urine and plasma, but their molecular weights, immunogenicity and even receptors are different from EPO. The content of such active proteins is very low, it is difficult to obtain enough pure products for quality research, and finally, it is found that impurities from an α₁-antitrypsin preparation of the human plasma has high activity for promoting generation of reticulocyte. Several high-activity proteins, obtained by biochemical and purifying means such as High Performance Liquid Chromatography (HPLC), are peptide fragments of different lengths in a precursor of the α₁-antitrypsin, and have carbohydrates of different lengths, and some components have few or no carbohydrate.

### Technical Solutions of Embodiments of the Present Invention

### 1. Purification of RGF

a, the α₁-antitrypsin preparation (A-9024) of Sigma Company is from human plasma, and has very high activity for promoting generation of the reticulocyte. Activity inspection adopts a mouse reticulocyte determining method of professor CHENG Yaqin, which is described as follows:

animals: ICR mice, weight: 18-22 kg, male

samples: diluting according to the activity of RGF, 5-20 μg/ml in common

method: dividing three mice in one group, intramuscularly injecting 0.06 ml, 0.12 ml and 0.24 ml, injecting for three days continuously, sampling eye socket blood on the fourth day, adding 7-8 drops into a tube with a lid filled with 20 μl 100 U/ml of heparin sodium, mixing uniformly, taking out 50 μl blood, dropwise adding on dried brilliant cresyl blue dye precoated at one end of a glass slide, fully mixing, and placing in a 37° C. wet box for 30 minutes; pushing single layer of cells on a second glass slide, after naturally drying, fixing with methyl alcohol, then redyeing through Wright's stain, dropwise adding 0.02 mol/LPBS (PH6.8), avoiding drying up, and washing the dye liquor with PBS or distilled water after 30 minutes;

reading a film by a high power oil immersion lens, where a nucleus substance in the reticulocyte can be dyed to be deep blue color dot-like, line-like, chain-like or cluster-like, while the red cells do not contain such substance; counting 500-1000 red cells in common and calculating the percentage of the reticulocyte; reading two films for each sample, averaging, then averaging again for three mice in each group, and subtracting the percentage of the reticulocyte in a control group (0.12 μl normal saline) to obtain the net increase percentage of the reticulocyte, defining every 1% of increase as one unit (U), and calculating specific activity by injected protein content (once), namely taking the rising unit stimulated by each mg of protein (U/mg) as the specific activity;

preparing the α₁-antitrypsin preparation into different concentrations with normal saline, taking 15 ICR male mice (weight: 18-22 g), dividing into 5 groups each of which contains 3 mice, intramuscularly injecting 0.12 ml normal saline into the control group, injecting 0.12 ml antitrypsin preparation of different concentrations into the rest grouped mice, injecting for three days continuously, sampling eye socket blood on the fourth day, dyeing by brilliant cresyl blue, and calculating the percentage of the reticulocyte in the red cells with the oil immersion lens, wherein a result shows that the activity for promoting the generation of the reticulocyte in the antitrypsin preparation is very high, and can serve as an initial material for further separation and purifying. The result is as shown in Table 1:

TABLE 1
Activity for promoting generation of reticulocyte
in α₁-antitrypsin preparation

| α₁-antitrypsin | | Net increase |
|---|---|---|
| preparation concentration | Reticulocyte % | reticulocyte % |
| 0 | 1.8 | |

| | | | |
|---|---|---|---|
| 2 | μg/ml | 8.2 | 6.4 |
| 5 | μg/ml | 6.6 | 4.6 |
| 20 | μg/ml | 16.45 | 14.65 |
| 80 | μg/ml | 1.8 | 0 |

The data indicates that the α₁-antitrypsin preparation indeed contains higher activity for promoting generation of reticulocyte.

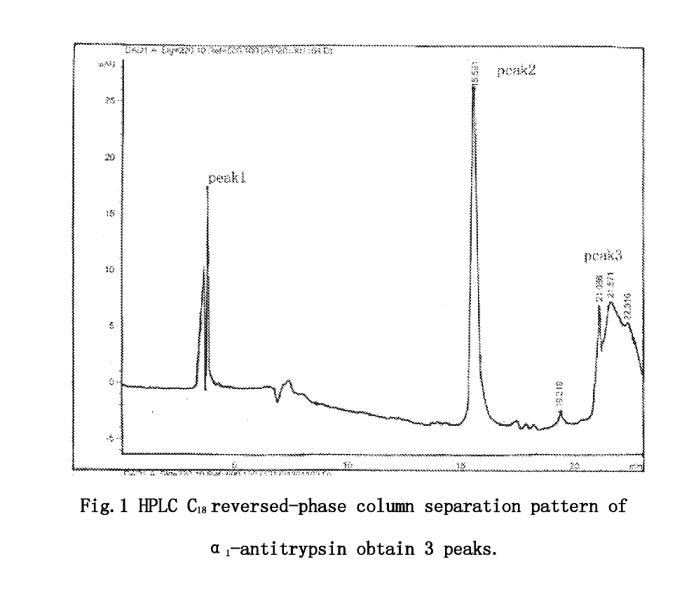

b, dissolving the α₁-antitrypsin into 2 mg/ml with PBS of 0.02 mol/L, performing neutral separating by an HPLC C₁₈ reversed-phase column to obtain 3 peak groups, wherein, through determining, only the 3rd peak group has activity, a C18 reversed-phase column separation pattern is as shown in FIG. 1 and a result is as shown in Table 2:

TABLE 2
Activities of respective parts obtained by separating the
α₁-antitrypsin through the HPLC C₁₈ reversed-phase column

| | | Net increase |
|---|---|---|
| Collected parts | Reticulocyte % | reticulocyte % |
| Control saline 0.12 ml | 2.4 | |
| Peak 1 20 μg/ml 0.12 ml | 3.3 | 0.9 |
| Peak 2 20 μg/ml 0.12 ml | 4.48 | 2.08 |
| Peak 3 20 μg/ml 0.12 ml | 8.6 | 6.2 |

c, increasing an eluting gradient of the peak 3 group with the C18 reversed-phase column again to obtain 10 collected parts, wherein an activity inspection result shows that the 8th collected part has the highest activity, the 7th and 9th collected parts have the activity second to that of the 8th collected part, and the other 7 collected parts have no activity. A separating pattern is as shown in FIG. 1, and an activity determining results are shown in Table 3:

TABLE 3
Activities of 7^{#}, 8^{#}, 9^{#} and 10^{#} parts in the 10 collected
parts obtained by separating the third peak for the second time

| | | Net increase |
|---|---|---|
| Collected parts | Reticulocyte % | reticulocyte % |
| Control saline 0.12 ml | 1.2 | |
| Peak 7^{#} 20 μg/ml 0.12 ml | 4.9 | 3.7 |
| Peak 8^{#} 20 μg/ml 0.12 ml | 8.3 | 7.1 |
| Peak 9^{#} 20 μg/ml 0.12 ml | 5.8 | 4.6 |
| Peak 10^{#} 20 μg/ml 0.12 ml | 4.1 | 2.9 |

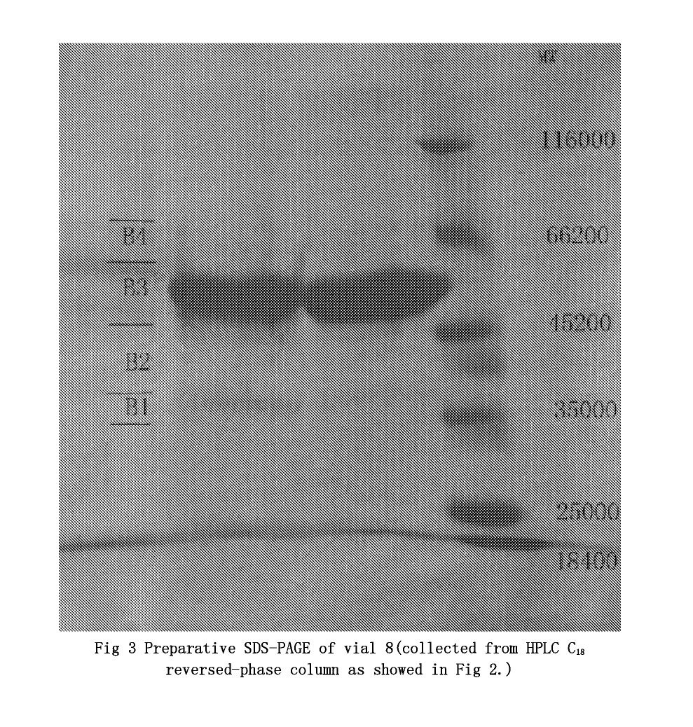

d, performing preparative SDS-PAGE (as shown in FIG. 3) on the eighth collected part, cutting a piece of gel, according to the position displayed by staining, for the undyed gel, cut first to fourth bands of gel, respectively triturating, extracting with water for 24 hours, extracting for three times continuously, combining supernate, determining activity after freeze-drying, wherein, only the bands 1, 2 and 3 extracting liquor show activity, the band 4 has no activity, a result is as follows:

TABLE 4
Activities of extracting liquors of respective bands
cut by electrophoresis and prepared by the activity
peak eighth sample after HPLC separation

| | | Net increase |
|---|---|---|
| Prepared electrophoresis bands | Reticulocyte % | reticulocyte % |
| Control saline 0.12 ml | 1.2 | |
| Band 1 20 μg/ml 0.12 ml | 10.7 | 9.2 |
| Band 2 20 μg/ml 0.12 ml | 8.1 | 6.6 |
| Band 3 10 μg/ml 0.12 ml | 7.2 | 5.7 |
| Band 4 20 μg/ml 0.12 ml | 1.7 | 0.2 |

then performing SDS-PAGE inspection after freeze-drying of the plurality of extracting liquors, wherein the bands 1 and 3 have clear dyed strips, the band 2 is high in activity but is close to the band 1, possibly has pollution, is fuzzily dyed, and is obvious not single bands, therefore, samples of the bands 1 and 3 are sent to the Suzhou ProtTech Inc. for sequencing, an identification result shows that the bands 1 and 3 are a precursor of the α₁-antitrypsin, we called the peptide fragment in such family as Reticulocyte Growth Factors (RGF family), and the sequence of the precursor is:

(SEQ ID NO: 1)
Met Pro Ser Ser Val Ser Trp Gly Ile Leu Leu Leu Ala Gly Leu Cys
Cys Leu Val Pro Val Ser Leu Ala Glu Asp Pro Gln Gly Asp Ala Ala
Gln Lys Thr Asp Thr Ser His His Asp Gln Asp His Pro Thr Phe Asn
Lys Ile Thr Pro Asn Leu Ala Glu Phe Ala Phe Ser Leu Tyr Arg Gln
Leu Ala His Gln Ser Asn Ser Thr Asn Ile Phe Phe Ser Pro Val Ser
Ile Ala Thr Ala Phe Ala Met Leu Ser Leu Gly Thr Lys Ala Asp Thr
His Asp Glu Ile Leu Glu Gly Leu Asn Phe Asn Leu Thr Glu Ile Pro
Glu Ala Gln Ile His Glu Gly Phe Gln Glu Leu Leu Arg Thr Leu Asn
Gln Pro Asp Ser Gln Leu Gln Leu Thr Thr Gly Asn Gly Leu Phe Leu
Ser Glu Gly Leu Lys Leu Val Asp Lys Phe Leu Glu Asp Val Lys Lys
Leu Tyr His Ser Glu Ala Phe Thr Val Asn Phe Gly Asp Thr Glu Glu
Ala Lys Lys Gln Ile Asn Asp Tyr Val Glu Lys Gly Thr Gln Gly Lys
Ile Val Asp Leu Val Lys Glu Leu Asp Arg Asp Thr Val Phe Ala Leu
Val Asn Tyr Ile Phe Phe Lys Gly Lys Trp Glu Arg Pro Phe Glu Val
Lys Asp Thr Glu Glu Glu Asp Phe His Val Asp Gln Val Thr Thr Val
Lys Val Pro Met Met Lys Arg Leu Gly Met Phe Asn Ile Gln His Cys
Lys Lys Leu Ser Ser Trp Val Leu Leu Met Lys Tyr Leu Gly Asn Ala
Thr Ala Ile Phe Phe Leu Pro Asp Glu Gly Lys Leu Gln His Leu Glu
Asn Glu Leu Thr His Asp Ile Ile Thr Lys Phe Leu Glu Asn Glu Asp
Arg Arg Ser Ala Ser Leu His Leu Pro Lys Leu Ser Ile Thr Gly Thr
Tyr Asp Leu Lys Ser Val Leu Gly Gln Leu Gly Ile Thr Lys Val Phe
Ser Asn Gly Ala Asp Leu Ser Gly Val Thr Glu Glu Ala Pro Leu Lys
Leu Ser Lys Ala Val His Lys Ala Val Leu Thr Ile Asp Glu Lys Gly
Thr Glu Ala Ala Gly Ala Met Phe Leu Glu Ala Ile Pro Met Ser Ile
Pro Pro Glu Val Lys Phe Asn Lys Pro Phe Val Phe Leu Met Ile Glu
Gln Asp Thr Lys Ser Pro Leu Phe Met Gly Lys Val Val Asn Pro Thr
Gln Lys.

### 2. Chemical Properties of RGF

In order to determining the chemical properties of the bands 1 and 3 prepared by SDS-PAGE, the gel bands displayed by staining were sent to the Suzhou ProtTech Inc. for determining the sequences of the N end and C end, a result shows that the band 1 (RTF-6 in a sequencing report of the Suzhou ProtTech Inc.) contains four components which are fragments of different lengths of the precursor of the α₁-antitrypsin, N end sites have slight difference, the C ends are same as the C ends of precursor, band 3 (RTF-8 in a report of the Suzhou ProtTech Inc.) contains only one component, the N end and C end are totally same as the bands 1-2 and contain 394 amino acids in total from the 25 site to the 418 site, the band 1 has a molecular weight of about 50000, which is 10000 larger than the band 3, it is known that the band 1 contains carbohydrate, the components in the band 1 are glycoproteins, the band 3 does not contain carbohydrate through speculation from the molecular weight, or contains extremely less carbohydrate.

The RGF family is a group of protein fragments with different lengths and different carbohydrate contents from the precursor of the α₁-antitrypsin. An amino acid sequence thereof is:

N end (band1-1):
(SEQ ID NO: 2)
Ala Glu Asp Pro Gln Gly Asp Ala Ala Gln Lys Thr
Asp Thr Ser His His Asp Gln Asp His Pro Thr Phe
Asn Lys
N end (band 1-2):
(SEQ ID NO: 3)
Glu Asp Pro Gln Gly Asp Ala Ala Gln Lys Thr Asp
Thr Ser
N end (1-3):
(SEQ ID NO: 4)
Pro Gln Gly Asp Ala Ala Gln Lys Thr Asp Thr Ser
His His Asp Gln Asp His Pro Thr Phe Asn Lys
N end (band 1-4):
(SEQ ID NO: 5)
Asp Ala Ala Gln Lys Thr Asp Thr Ser His His Asp
Gln Asp His Pro Thr Phe Asn Lys
C fragment:
(SEQ ID NO: 6)
Val Val Asn Pro Thr Gln Lys

### 3. Biological Properties of RGF

a, therapeutic action of the RGF for aplastic anemia mice, this experiment is entrusted to the Pharmacology Teaching and Research Department of the Institute of Traditional Chinese medicine, Jiangsu,

modeling: Injecting 100 mg/kg of cyclophosphamide ip into male mice of 18-22 g for three continuous days

test: the mice are divided into 5 groups and EPO is domestic (NING Hongxin). The RGF is the produc prepaed in this laboratory, and a result of RBC and WBC examining after continuous injecting for 8 days is as shown in Table 5:

TABLE 5
Therapeutic action of the RGF to aplastic anemia mice

| | Mouse | | | | | |
|---|---|---|---|---|---|---|
| | sample | | Result | | Red cell | Leukocyte |
| Groups | number | Dosage | RBC | WBC | promotion % | promotion % |
| Blank control | 20 | Normal saline | 487 ± 149.3 | 190 ± 71.8 | | |
| Aplastic | 20 | Normal saline | 305 ± 102.5 | 115 ± 36.5 | | |
| anemiamodel | | | | | | |
| group | | | | | | |
| EPO group in | 20 | 333 U/kg | 429 ± 94.8 | 201 ± 79.6 | 40.66 | 77.78 |
| model group | | | | | | |
| RGF group in | 20 | 167 μg/kg | 442 ± 105.1 | 195 ± 81.1 | 40.92 | 69.57 |
| model group | | | | | | |
| EPO group + | 20 | ½EPO ± ½RPF | 516 ± 108.4 | 233 ± 104.5 | 69.18 | 102.61 |
| RGF group in | | | | | | |
| model group | | | | | | |

The cyclophosphamide can obviously cause anemia to the mice, gneration of the red cells can be promoted by injecting the EPO or RGF, for example, the therapeutic action, achieved by half amount of EPO and half amount of RGF, for co-treating the generation of the red cells is better than the therapeutic action achieved by single whole amount of the RGF or EPO, an obvious synergistic effect is achieved, and it should be noted that the RGF or EPO further has a promotion action for the generation of leukocytes, and have the same synergistic effect.

b, Synergistic Effect of the RGF and EPO for Generation of Mouse Reticulocyte

animals: 30 ICR mice, male, weight: 18-22 g, 10 groups, 3 mice in each group

experiment: preparing the RGF and EPO into three following concentrations: (a) RGF5 μg/ml; (b) EPO (rhuEPO of Amgen Company) 18 U/ml; (c) balanced mixing of the RGF and EPO; injecting the RGF and EPO with the concentrations of the a, b and c into the mice in each group by 0.06 ml, 0.12 ml and 0.24 ml for continuous 3 days, sampling eye socket blood on the fourth day, smearing, dyeing, and examining the increase percentage of the reticulocyte with a microscope, wherein a result is as shown in Table 6:

TABLE 6
Synergistic effect of RGF and EPO (Amgon Company)

| Injecting | Net increase reticulocyte % | | |
|---|---|---|---|
| dosage (each | | | EPO9 U/ml |
| mouse every day) | EPO (18 U/ml) | RGF (5 μg/ml) | RGF2.5 μg/ml |
| 0.06 ml | 4.8% | 4.9% | 5.3% |
| 0.12 ml | 6.2% | 6.6% | 7.2% |
| 0.24 ml | 7.4% | 7.8% | 11.3% |

The RGF and EPO both have well activity for promoting the generation of the reticulocyte, 18 U/ml of EPO and 5 μg/ml of RGF are very close in activity, after the EPO and the RGF are mixed in the same amount, an obvious synergistic effect is generated, especially, the test group injected with the 0.24 ml is higher than the test group injected with the 0.24 ml of the EPO or RGF by about 50% in reticulocyte increase, then is this phenomenon possibly the false appearance caused by the generation of the mouse EPO caused by the RGF in vivo, for this purpose, we design a process that after the RGF of different dosages is injected to the mice for 3 days, the EPO level in blood of the mice is determined by monoclonal antibody to compare with the normal saline control group, the EPO level is not increased, so that such possibility is excluded. It is inferred that the EPO and the RGF act in different development stages of the red cells, under a relay action of the EPO and the RGF, the speed of the red cells is naturally increased. This hypothesis is verified in follow fluorescence labeling experiment.

The synergistic effect of the RGF and EPO occurs in the two experiments, and this phenomenon has great significance in treatment of the severe anemia and indicates a potential of more reasonably and more effectively treating the severe anemia.

c, Fluorescence Labeling Experiment

labeling the EPO with fluorochrome rhodamine (RB200), wherein the EPO is red under the observation of a fluorescence microscope, labeling the RGF with fluorochrome FITC, wherein the RGF is green under the observation of the fluorescence microscope, sampling about 2 ml of bone marrow cells from longs bones of four mice, centrifuging at 1000 r/m for four minutes, pouring off supernatant, resuspending in 6 ml of Hank'S liquid, separating through a lymphocyte separating medium, and taking and centrifuging interface cells; diluting the cells with RPMI1640 into 1.8×10⁶/ml of cell suspension, taking 5 ml of cell suspension, centrifuging, resuspending in 0.3 ml of PBS, adding 60 μl of FITC-RGF and 60 μl of RB200-EPO, placing in a refrigerator for 30 minutes, wherein, by observing under the fluorescence microscope, most cells are doubly labeled, a few cells are singly labeled by FITC-RGF, but there are no cells labeled by RB200-EPO. This phenomenon prompts that the RGF is used in an earlier stage of a cell development process, an EPO receptor does not occur till the cells are developed to the next stage under the promotion of the RGF, therefore, the dual-receptor cells on which the RGF and EPO show at the cells occur.

### Beneficial Effects of Embodiments of the Present Invention

Differentiation and maturation of the red cells are a multi-stage multifactor-participated complicated process, however, a second factor for promoting the generation of the red cells is not found for last 20 years, so that the finding of the reticulocyte growth factor has great significance. When the reticulocyte growth factor and EPO are combined in use, the effect is better than that when the reticulocyte growth factor or EPO is singly used. It is indicated that different development stages of the red cells need different induction and promotion of different factors, which is a new start for us to learn the total development process of the red cells, and treat the severe anemia. Embodiments of the present invention helps to explain the synergistic effect of the RGF and EPO occurring in normal mice and aplastic anemia mice. Because they act in different development stages of the red cells, and bone marrow cells generate more EPO receptors under the action of the RGF, and thus are developed to dual-receptor cells, the EPO then continuous acts, and the better effect can be understood under a relay action. The invention develops a new medicine for treating the severe anemia.

### BRIEF DESCRIPTION

FIG. 1 is a chromatography pattern of an α₁-antitrypsin separated by an HPLC C₁₈ reversed-phase column in first separation, wherein 3 collected parts are obtained from the first separation, and the 3rd part has activity.

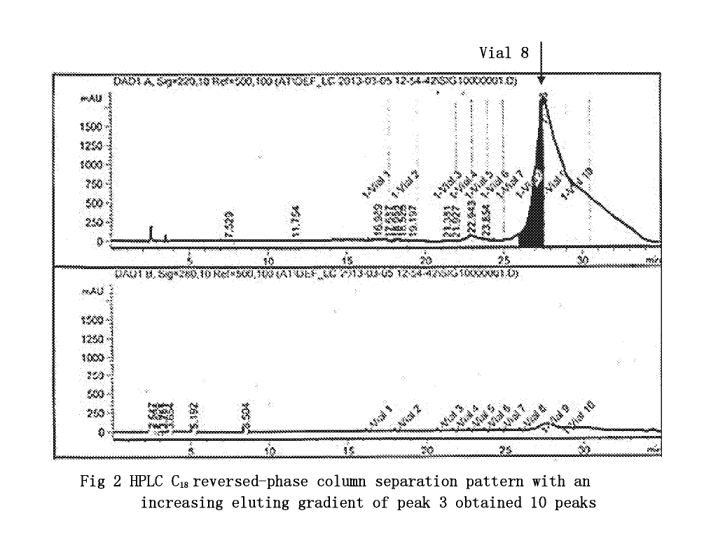

FIG. 2 is 10 collected parts obtained by the HPLC C₁₈ reversed-phase column in second separation larger than the first separation by gradient, wherein the eighth peak collected part pointed by the arrow has higher activity.

FIG. 3 is an SDS-PAGE diagram of an eighth part in the ten collected parts in the second HPLC separation, wherein a molecular weight is about 50000, the part dyed with the deepest color is band 1 (RGF-B1), the molecular weights of about 40000 are called as band 3 (RGF-B3), the part between the B1 and B3 is called as RGF-B2, and the parts above B1 have no activity.

### DETAILED DESCRIPTION

1. Taking two bottles of α₁-antitrypsin, dissolving in pH7.0 0.02 MOL/L of PBS with the concentration of 2.5 mg/ml, performing neutral separation by an High Performance Liquid Chromatography (HPLC) C18 column, and obtaining 3 peak groups, wherein, as shown in FIG. 1, the 3rd peak group shows a strong action for promoting the generation of reticulocyte;

2. re-separating the third peak group, but pulling the separated eluting gradient, and obtaining 10 collected parts, wherein only the 7th, 8th and 9th collected parts show the activity for promoting the generation of the reticulocyte, the 8th collected part has the highest activity as shown in FIG. 2 and FIG. 3; performing preparative SDS-PAGE on the 8th collected part, longitudinally cutting a piece of gel, dyeing by Coomassie Brilliant Blue to determine the position of proteins, wherein a deeper and wider band occurs at the position of the molecular weight of about 50000; cutting the corresponding gel block as band 1, cutting a clear band at the position of the molecular weight of about 40000 as band 3, wherein, 3-4 dyed bands between the band 1 and band 3 are fuzzy, one of the bands is close to number 1, and is called as band 2, and the band between the molecular weights of 50000-68000 is called as band 4; crushing the gel of the four parts with glass rods individually, extracting for 24 hours with water, sucking out supernatant, and repeating for three times; combining and freeze-drying the extracting liquors obtained at four times on the 5th day, and dissolving with a small amount of water and then determining the activity, wherein a result shows that the extracting liquors of the bands 1, 2 and 3 have high activity, and the band 4 has no activity, and the result is as shown in table 3;

3. performing SDS-PAGE on the extracting liquors of the bands 1, 2 and 3, wherein the bands 1 and 3 have clear strips after staining, the band 2 has fuzzy strips, some strips are very close to the band 1 in position, therefore, the dyeing gel of the bands 1 and 3 are sent to the ProtTech Inc. for sequencing, a determining result shows that the bands 1 and 3 are parts of a precursor of the α₁-antitrypsin and a result is as shown in the amino acid sequence;

4. in order to determine the chemical properties of the bands 1 and 3, the SDS-PAGE, drying gel blocks of the band 1 and band 3 are sent to the Suzhou ProtTech Inc. for sequencing, and a result is as shown in attachment 2. The N end and C end sequencing result shows that four components contained in the band 1 are fragments of different lengths in the precursor of the α₁-Antitrypsinm, the precursor is composed of 418 amino acids, these fragments have different start points of the N ends, and are respectively 24, 25, 27 and 30 site amino acids; the C ends are same as the tail ends of the precursor. The band 3 contains single component, is totally same as the band 1-2 in structure and is composed of 394 amino acids in total from the 25 h site to the 418th site. But the molecular weight is smaller than band 1 that contains carbohydrate, and based on the molecular weight, the band 3 contains no or extremely less carbohydrate;

5. the sequence of the precursor of the α₁-Antitrypsin is known, the molecular weight of antitrypsin is known as 55000-56000, the bands 1 and 3 are both smaller than the precursor in molecular weight, and plural components between the molecular weights of 50000-40000 also show activity. It is estimated that the precursor of the Antitrypsin can be degraded into a group of peptide fragments in different lengths, and these peptide fragments also have carbohydrate chains of different lengths or no carbohydrate. It is still unknown yet that whether the active protein groups composed of the peptide fragments have different action sites, but all the components of the antitrypsin family participate in the action of regulating the generation of the red cells in organism together with the EPO.

1. A reticulocyte growth factor (RGF), having an amino acid sequence that is a fragment of SEQ ID NO: 1, wherein SEQ ID NO: 1 is a 418 amino acid precursor of α₁-antitrypsin, the RGF and SEQ ID NO: 1 belonging to an RGF family, the RGF is selected from the group consisting of SEQ ID NO: 2, SEQ ID NO: 3, SEQ ID NO: 4, SEQ ID NO: 5 and SEQ ID NO: 6, wherein all glycoprotein and non-glycoprotein with at least 70% of SEQ ID NO: 1 or the RGF fragment thereof belong to the RGF family.

2. A method for preparing the reticulocyte growth factor of claim 1, comprising the steps of:
dissolving α₁-antitrypsin preparation from human plasma into 2.5 mg/ml with PBS of 0.02 mol/L;
separating the α₁-antitrypsin preparation through an HPLC C_{18F} reverse-phase column to obtain 3 protein peak groups, wherein through activity determining, a third peak group of the 3 protein peak groups has activity of promoting growth of a reticulocyte
separating the third peak group through the HPLC C_{18F} reverse-phase column again, wherein 10 protein collected parts are obtained when an eluting gradient is increased, and the 8th part of the 10 protein collected parts has higher activity through activity determining;
performing preparative SDS-PAGE on the 8th part, cutting a portion of gel of the SDS-PAGE with the RGF protein and crushing the gel, repeatedly extracting with water, detecting purity and activity after concentration, and sequencing.

3. An application of the reticulocyte growth factor of claim 1 for preparing a medicine for treating anemia.

4. The application of the reticulocyte growth factor according to claim 3 for preparing the medicine for treating anemia, wherein the reticulocyte growth factor and erythropoietin are combined for use, having a synergistic effect.


SEQUENCE LISTING (6 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQDTKSPLFMGKVVNPTQK

SEQ ID NO: 2 - protein, 26 aa, Artificial Sequence
  N end band 1-1, Protein fragment of alpha-1-antitrypsin
AEDPQGDAAQKTDTSHHDQDHPTFNK

SEQ ID NO: 3 - protein, 14 aa, Artificial Sequence
  N end band 1-2, Protein fragment of alpha-1-antitrypsin
EDPQGDAAQKTDTS

SEQ ID NO: 4 - protein, 23 aa, Artificial Sequence
  N end band 1-3, Protein fragment of alpha-1-antitrypsin
PQGDAAQKTDTSHHDQDHPTFNK

SEQ ID NO: 5 - protein, 20 aa, Artificial Sequence
  N end band 1-4, Protein fragment of alpha-1-antitrypsin
DAAQKTDTSHHDQDHPTFNK

SEQ ID NO: 6 - protein, 7 aa, Artificial Sequence
  C fragment - Protein fragment of alpha-1-antitrypsin
VVNPTQK